(19) United States
(12) Patent Application Publication
(10) Pub. No.: US 2017/0340348 A1
(43) Pub. Date: Nov. 30, 2017

(54) TROCAR TIP PROTECTOR

(71) Applicant: Covidien LP, Mansfield, MA (US)

(72) Inventor: Ramiro Cabrera, Cheshire, CT (US)

(21) Appl. No.: 15/467,346

(22) Filed: Mar. 23, 2017

Related U.S. Application Data
(60) Provisional application No. 62/343,237, filed on May 31, 2016.

(51) Int. Cl. A61B 17/34; A61B 17/072; A61B 17/115

(52) U.S. Cl. CPC A61B 17/34; A61B 17/1155; A61B 17/072; A61B 2017/07278; A61B 2017/07228

(57) ABSTRACT

A protection device includes an elongate body having a distal portion defining a flange, and a proximal portion including a plurality of legs extending proximally from the distal portion. When a distal surface of the flange is positioned on a horizontal surface, the plurality of legs is oriented in a vertical orientation.

### CROSS-REFERENCE TO RELATED APPLICATIONS

This application claims the benefit of and priority to U.S. Provisional Patent Application No. 62/343,237 filed May 31, 2016, the entire disclosure of which is incorporated by reference herein.

### BACKGROUND

### 1. Technical Field

The present disclosure is directed to embodiments of a trocar tip protector for shielding the trocar tip of a surgical stapling device during periods of non-use between surgical procedures.

### 2. Background of Related Art

In an effort to reduce the cost of medical procedures, surgical stapling devices that once were disposable are now being constructed to be reusable. In order to reuse a surgical stapling device, the stapling device must be re-sterilized. This necessarily requires transport of a used surgical stapling device to an apparatus for re-sterilizing the used surgical stapling device.

Current circular surgical stapling devices typically include a trocar having a sharp tip. In order to minimize the likelihood of transferring blood-borne diseases such as hepatitis B (HBV), hepatitis C (HCV), and human immunodeficiency virus (HIV) to healthcare workers or healthcare personnel caused by contact with the trocar tip, a need exists in the art for a device for shielding the trocar tip. It would also be desirable to provide such a device that facilitates sterilization of the trocar with the device secured to the trocar of the surgical stapling device.

### SUMMARY

The present disclosure is directed to a protection device including an elongate body that includes a distal portion defining a flange and a proximal portion including a plurality of legs extending proximally from the distal portion. When a distal surface of the flange is positioned on a horizontal surface, the plurality of legs is oriented in a vertical orientation.

In aspects, the distal portion may include a tubular portion positioned between the enlarged flange and the plurality of legs. The tubular portion may have an outer surface defining an annular groove. In embodiments, the annular groove is configured to allow grasping of the protection device.

In some aspects, a distal surface of the enlarged flange may include a depression formed therein.

In certain aspects, the protection device may be formed from a material suitable for sterilization.

In some aspects, each leg of the plurality of legs may terminate in an inwardly extending barb where each barb is configured to engage a trocar of a surgical device.

In aspects, a proximal portion of each leg of the plurality of legs may include an outer surface including a proximal flange that is dimensioned and configured to inhibit the protection device form being pulled within a surgical device when a trocar of the surgical device is retracted.

In certain aspects, each leg of the plurality of legs may include an inner surface defining an annular groove configured to engage a trocar of a surgical device.

In aspects, the flange may be circular.

In some aspects, the flange may be rectangular.

In certain aspects, the tubular portion may define a bore that extends through at least a portion of the tubular portion.

Another aspect of the present disclosure is directed to a protection device including an elongate body and a lever. The elongate body includes a distal portion defining a flange and a proximal portion including a proximal face that defines a hole configured to receive a trocar of a surgical device. The lever is rotatably secured to the elongate body and is configured to releasably engage a trocar of a surgical device.

In aspects, the protection device may further include a biasing element interposed between the lever and the elongate body. The biasing element biases the lever into engagement with a trocar of a surgical device.

In certain aspects, the flange may include an outer dimension that is larger than the outer dimension of the surgical device.

### BRIEF DESCRIPTION OF THE DRAWINGS

The above and other aspects, features, and advantages of embodiments of the presently disclosed trocar tip protector will become more apparent in light of the following detailed description when taken in conjunction with the accompanying drawings in which:

FIG. 1 is a front, perspective view, of embodiments of the presently described trocar tip protector provided in accordance with the present disclosure, shown as being coupled to a powered surgical stapling device;

FIG. 2 is the enlarged view of the distal end of the powered surgical stapling device shown in FIG. 1 with the trocar tip protector removed from a trocar of the powered surgical stapling device;

FIG. 3 is an enlarged view of the area of detail indicated in FIG. 1;

FIG. 4 is a side, perspective view, of the trocar tip protector shown in FIG. 1 positioned adjacent a trocar of the powered surgical stapling device of FIG. 1;

FIG. 5 is a side, perspective view, of the trocar tip protector of FIG. 4 coupled to the trocar of the powered surgical device of FIG. 1;

FIG. 6 is a side, perspective view, of the trocar tip protector shown in FIG. 4;

FIG. 7 is a front, perspective view, of the trocar tip protector of FIG. 4;

FIG. 8 is a side, perspective view, of an extension assembly of the powered surgical stapling device of FIG. 1 and the trocar tip protector shown in FIG. 4 with parts separated;

FIG. 9 is a cross-sectional view taken along section line 9-9 of FIG. 7;

FIG. 10 is a cross-sectional view taken along section line 10-10 of FIG. 9;

FIG. 11 is a side, perspective view, of another embodiment of a trocar tip protector provided in accordance with the present disclosure;

FIG. 12 is a cross-sectional view taken along section line 12-12 of FIG. 11;

FIG. 13 is a side, cross-sectional view of the trocar tip protector of FIG. 11 coupled to the trocar of the powered surgical stapling device of FIG. 1;

FIG. 14 is a side, cross-sectional view of the trocar tip protector of FIG. 11 coupled to the trocar of the powered surgical stapling device of FIG. 1, shown with a circular loading unit attached to the powered surgical stapling device of FIG. 1;

FIG. 15 is a side, perspective view, of yet another embodiment of a trocar tip protector provided in accordance with the present disclosure;

FIG. 16 is a front, perspective view of the trocar tip protector of FIG. 15;

FIG. 17 is a side, perspective view, of the trocar tip protector of FIG. 15 shown with parts separated;

FIG. 18 is a cross-sectional view taken along section line 18-18 of FIG. 15;

FIG. 19 is a side, cross-sectional view, of the trocar tip protector of FIG. 15 shown partially coupled to the trocar of the powered surgical stapling device of FIG. 1;

FIG. 20 a side, perspective view, of the trocar tip protector of FIG. 15 coupled to the trocar of the powered surgical stapling device of FIG. 1;

FIG. 21 is a cross-sectional view taken along section line 21-21 of FIG. 20; and

FIG. 22 is a perspective view of a manual surgical stapling device suitable for use with a trocar tip protector in accordance with the present disclosure.

### DETAILED DESCRIPTION OF EMBODIMENTS

Embodiments of the presently disclosed trocar tip protector are now described in detail with reference to the drawings in which like reference numerals designate identical or corresponding elements in each of the several views. As used herein, the term “healthcare personnel” refers to a doctor, a nurse, or any other care provider and may include support personnel and/or technicians. Throughout this description, the term “proximal” will refer to the portion of the device or component thereof that is closer to the healthcare personnel and the term “distal” will refer to the portion of the device or component thereof that is farther from the healthcare personnel. Additionally, in the drawings and in the description that follows, terms such as front, rear, upper, lower, top, bottom, vertical, and similar directional terms are used simply for convenience of description and are not intended to limit the disclosure. In the following description, well-known functions or constructions are not described in detail to avoid obscuring the present disclosure in unnecessary detail.

In order to address the problems detailed above, the present disclosure relates to trocar tip protection devices capable of being releasably coupled to the trocar of the surgical stapling devices. The trocar tip protection devices detailed herein include an elongate body capable of receiving the trocar and an enlarged flange capable of maintaining the trocar tip protection device in an upright position. Once placed upon a surface to maintain the trocar tip protection device in an upright position, healthcare personnel are able to releasably couple the trocar of the powered surgical stapling device to the trocar tip protection device. Additionally, some embodiments of the trocar tip protection device include features that inhibit the trocar tip protection device from being pulled into an extension assembly of the powered surgical stapling device when the trocar is withdrawn. The trocar tip protection devices detailed herein enable healthcare personnel to easily shield the sharp distal tip of the trocar during transport and/or a sterilization process, minimizing the likelihood of transferring blood-borne diseases to healthcare personnel.

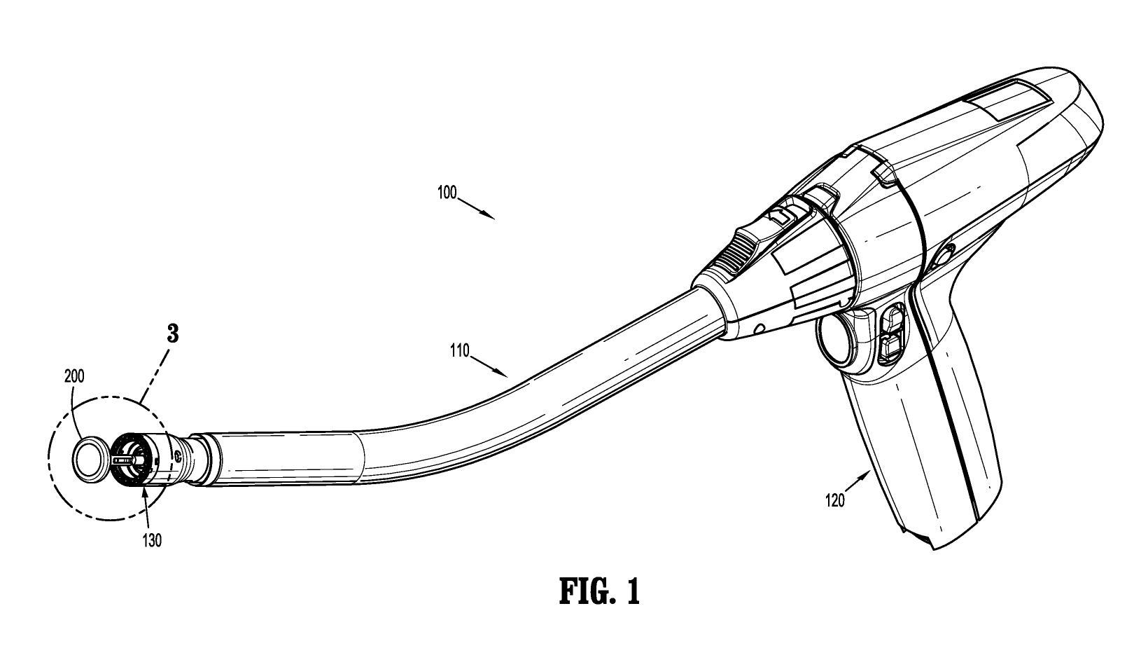

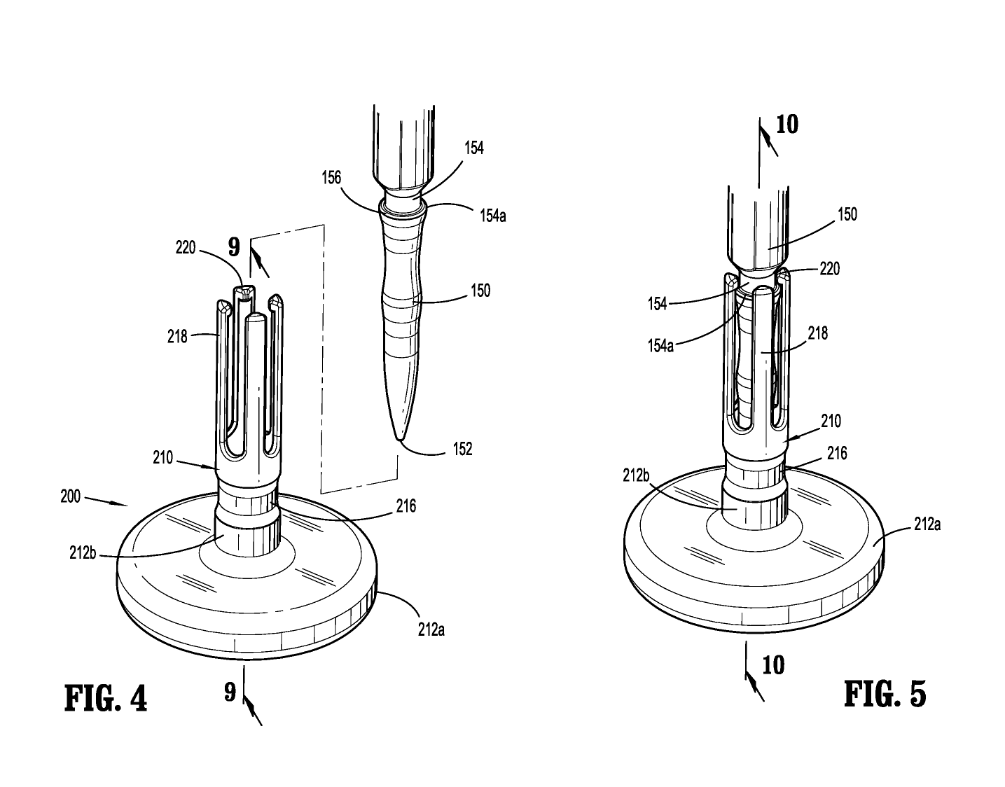

With reference to FIG. 1, a powered surgical stapling device (surgical instrument) suitable for use with the presently described trocar tip protector (protection device) 200 (FIG. 4) is illustrated and generally identified by reference numeral 100. Powered surgical stapling device 100 includes an extension assembly 110 and a handle assembly 120 configured for selective connection with a proximal end of the extension assembly 110. The extension assembly 110 has a distal end that is configured for selective connection with a circular loading unit 130 and an anvil assembly (not shown), for applying a circular array of staples (not shown) to tissue (not shown). For a detailed description of the structure and function of an exemplary powered surgical stapling device, please refer to commonly owned U.S. Pat. No. 9,055,943 to Zemlok et al., filed on May 31, 2012, the entire contents of which are incorporated by reference herein.

It is contemplated that any suitable loading unit, anvil assembly, and adapter assembly may be utilized with extension assembly 110. Exemplary loading units and anvil assemblies are described in commonly owned U.S. Pat. No. 8,590,763 and U.S. Patent Application Publication No. 2015/0108201, filed Oct. 17, 2013 and U.S. Patent Application Publication No. 2015/0190133, filed Jan. 7, 2014, the entire contents of each of which are hereby incorporated by reference herein.

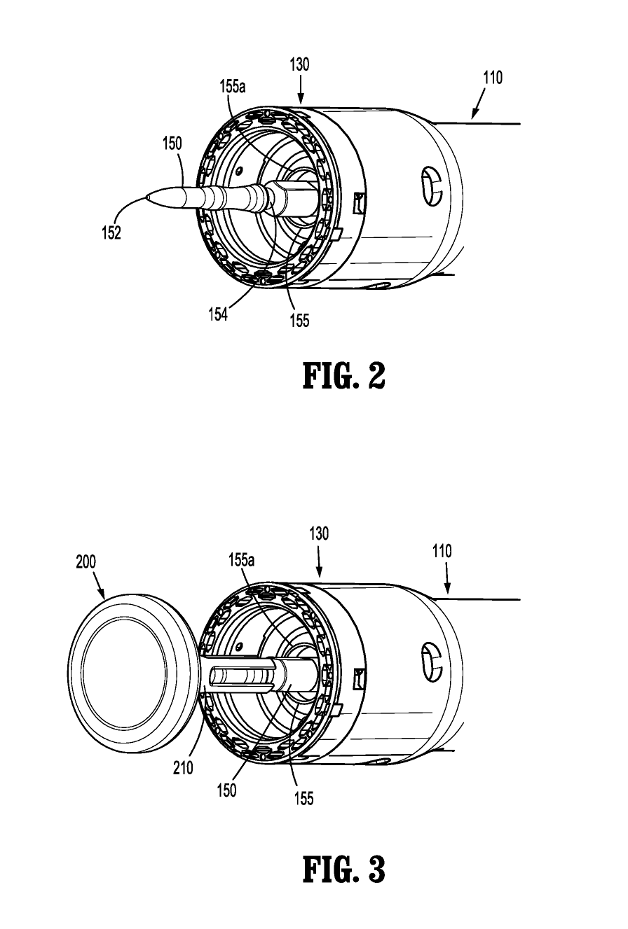

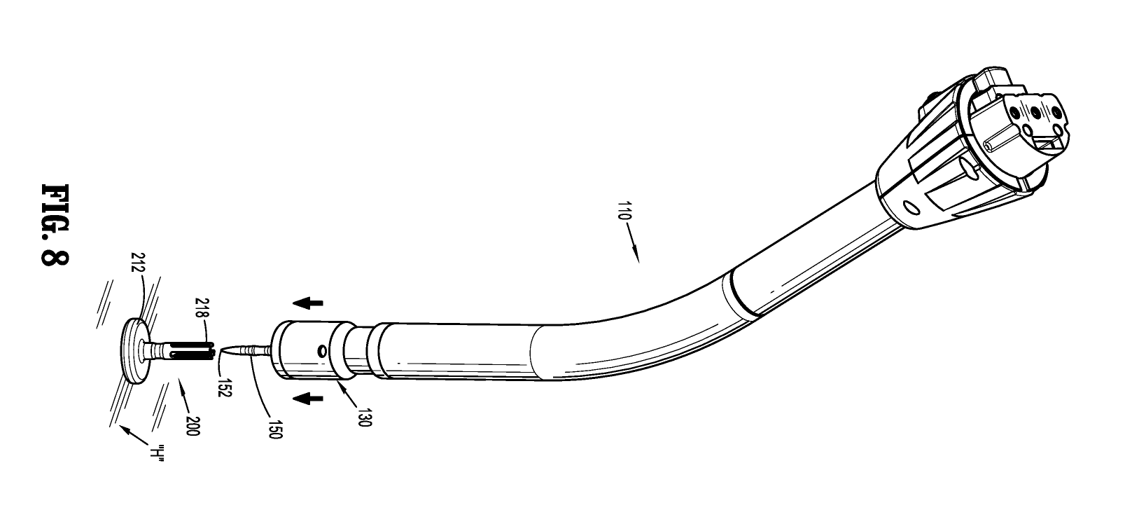

Referring also to FIGS. 2-5 extension assembly 110 includes a trocar 150 that extends from the distal end 155a of a housing 155 of the circular loading unit 130. The trocar 150 defines a generally cylindrical configuration having a pointed tip 152. A proximal portion of the trocar 150 includes an annular groove 154 defined within an outer surface thereof and forming a proximal facing lip 154a (FIG. 7) adapted to engage a suitable anvil assembly (not shown) as described in detail below.

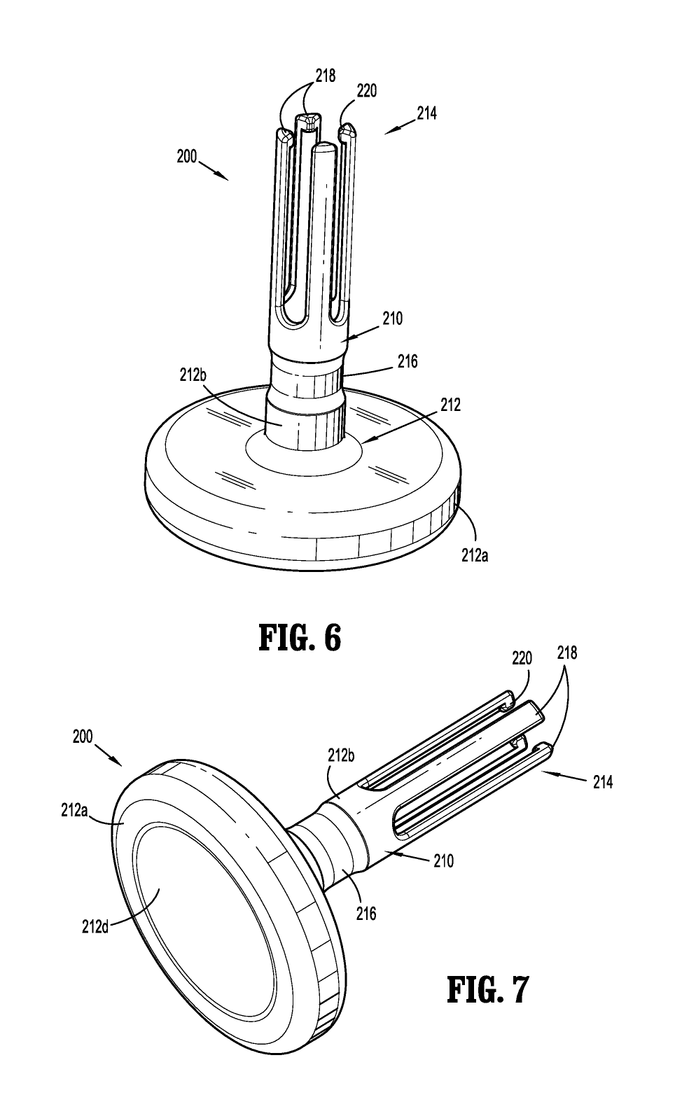

FIGS. 3-7 illustrate an exemplary embodiment of the trocar tip protector 200 (protection device) provided in accordance with the present disclosure. The trocar tip protector 200 includes an elongate body 210 having a distal portion 212 and a proximal portion 214. Although the elongate body 210 is shown as generally including a circular cross-section, it is contemplated that the elongate body 210 may include any suitable shape, such as square, rectangular, octagonal, or the like. The distal portion 212 includes a flared distal end defining a flange 212a (FIG. 4) and a tubular portion 212b extending proximally from the flange 212a. The tubular portion 212b defines a bore 212c. Although the flange 212a and the tubular portion 212b are shown as generally including a circular shape, it is contemplated that the flange 212a and/or the tubular portion 212b may include any suitable shape such as square, rectangular, octagonal, oval, or the like. The flange 212a includes an outer diameter greater than that of the elongate body 210 and the circular loading unit 130 (FIG. 3). In embodiments, the flange 212a provides sufficient surface area for the trocar tip protector 200 to rest with the body 210 extending upwardly when placed on a horizontal surface “H” (e.g., table, countertop, etc., FIG. 8). In this manner, healthcare personnel may push the extension assembly 110 towards the trocar tip protector 200 such that the protruding pointed distal tip 152 of the trocar 150 is received in the body 210, thereby inhibiting healthcare personnel from contacting the pointed distal tip 152 of the trocar 150 (FIG. 6). The large surface area of the flange 212a provides a high degree of stability and reduces the likelihood that the trocar tip protector 200 will move or wobble while healthcare personnel are inserting the pointed distal tip 152 of the trocar 150 into the body 210 of the trocar tip protector 200, as will be described in further detail hereinbelow.

As best illustrated in FIG. 7, the flange 212a includes a distal surface 212d that defines a depression, although the distal surface 212d may include any suitable shape, such as convex, planar, or the like. The elongate body 210 defines an outer surface including a recess 216. The recess 216 is configured and adapted to enable healthcare personnel to easily grasp the elongate body 210. Although shown as generally having a circular shape, it is contemplated that the recess 216 may include any suitable shape enabling ease of grasping. The proximal portion 214 of the trocar tip protector 200 includes a plurality of distally extending legs 218 that terminate in an inwardly extending barb 220. The barb 220 of each of the legs 218 extends radially inward such that the barbs 220 may releasably be received within the annular groove 154 of the trocar 150 (FIG. 5). In this manner, the barbs 220 are prohibited from passing distally over the proximally facing lip 154a of the annular groove 154 of the trocar 150 without the application of external force (i.e., the legs 218 deflect outward and over the proximally facing lip 154a). The trocar tip protector 200 advances over the trocar 150 until the pointed distal tip 152 of the trocar 150 is positioned within the body 210 of the trocar tip protector 200, thereby inhibiting healthcare personnel from contacting the sharp distal tip 152 of the trocar 150. Although the trocar tip protector 200 is generally shown as having four legs 218, it is contemplated that any suitable number of legs 218 may be employed on the trocar tip protector 200 to enable the trocar tip protector 200 to be releasably coupled to the trocar 150. For example, the trocar tip protector 200 may include two, three, five, six, or more legs. As can be appreciated, the trocar tip protector 200 may be constructed of any suitable material of sufficient strength and capable of withstanding a sterilization process, including resilient materials that permit deflection of the plurality of legs 218, e.g., metallic, polymeric, or composite materials.

With reference to FIGS. 8-10, after a procedure is performed on a patient and the loading unit 130 has been fired, the extension assembly 110 may be removed from handle assembly 120 (FIG. 1), the loading unit 130 is removed from the extension assembly 110, and the anvil assembly (not shown) is removed from the trocar 150, thereby exposing the pointed distal tip 152 of the trocar 150. The order that the different components are removed from the stapling device 100 is entirely within the discretion of healthcare personnel.

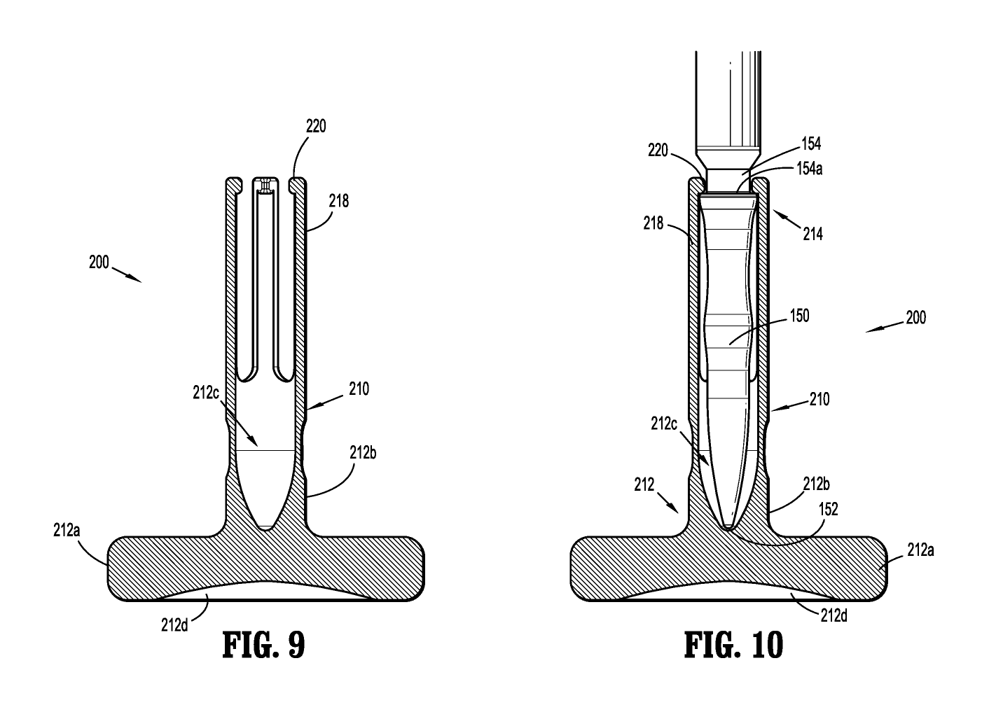

To secure the trocar tip protector 200 about the trocar 150, the trocar tip protector 200 is placed on a horizontal surface “H” (FIG. 8), such as a table, countertop, surgical tray, or the like, such that the distal surface 212d (FIG. 9) of the flange 212a is oriented in a downward facing orientation (i.e., the distal surface 212d is placed on the horizontal surface) and the plurality of legs 218 extend upward in a vertical orientation. Next, the extension assembly 110 is placed in a vertical orientation such that the trocar 150 is pointing in a downward direction towards the trocar tip protector 200 (FIG. 8). The extension assembly 110 is advanced (i.e., lowered) toward the trocar tip protector 200 such that the trocar 150 is received between the legs 218 of the trocar tip protector 200, and thereafter, bore 212c of the tubular portion 212b. As the trocar 150 is moved between the legs 218, the barbs 220 on the proximal ends of the legs 218 slide along the trocar 150 and the legs 218 are deflected outwardly. As the extension assembly 110 is further advanced toward the trocar tip protector 200, the barbs 220 of the trocar tip protector 200 pass over and are received within the annular groove 154 of the trocar 150 (FIG. 10). When this occurs, the legs 218 move inwardly to position the barbs 218 within the annular groove 154.

The trocar tip protector 200 is dimensioned such that when the barbs 220 of the trocar tip protector 200 are received in the annular groove 154 of the trocar 150 (FIG. 10), the pointed distal tip 152 of trocar 150 is positioned within the elongate body 210 to shield the pointed distal tip 152 of the trocar 150 from accidental contact by healthcare personnel during transport or during a sterilization procedure. In this manner, healthcare personnel are protected from puncture wounds or abrasions that may result from contact with the pointed distal tip 152.

The extension assembly 110, with the trocar tip protector 200 secured thereto, may be placed into a suitable sterilization chamber. After the sterilization process is complete, the extension assembly 110, with the trocar tip protector 200 remaining attached thereto, may be removed from the sterilization chamber and packaged or stored until a loading unit 130 is ready to be installed on the distal end of the extension assembly 110. At this point, the trocar tip protector 200 may be removed from the trocar 150 to facilitate attachment of the loading unit 130 to the extension assembly 110 (FIG. 1). The large diameter of the flange 212a of the trocar tip protector 200 provides a greater surface area on which healthcare personnel may grasp when removing the trocar tip protector 200 from the trocar 150.

In some embodiments, rather than being placed on a suitable surface as described above, the trocar tip protector 200 may be manually grasped and advanced over the trocar 150 of the extension assembly 110. In this manner, healthcare personnel would follow the above described process, except that the extension assembly 110 and the trocar tip protector 200 are moved in relation to each other. The remaining steps detailed above may then be followed.

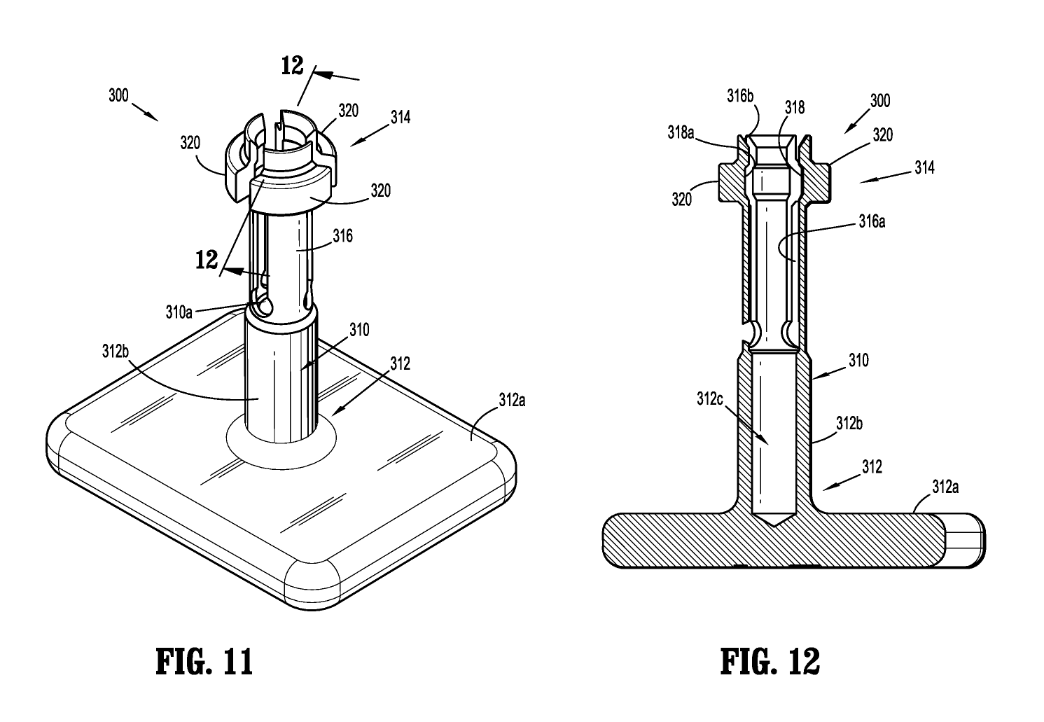

Referring now to FIGS. 11-14, another embodiment of a trocar tip protector (protection device) provided in accordance with the present disclosure is illustrated and generally identified by reference numeral 300. The trocar tip protector 300 includes an elongate body 310 including a distal portion 312 and a proximal portion 314. In embodiments, the elongate body 310 may have a circular cross-section, although other configurations are also contemplated such as square, rectangular, octagonal, or the like. The distal portion 312 includes a flared distal end defining a flange 312a and a tubular portion 312b extending proximally from the flange 312a. The tubular portion 312b defines a bore 312c. The flange 312a has a rectangular shape with an outer perimeter having a greater width than that of the elongate body 310 and the loading unit 130 (FIG. 14). The rectangular configuration of the flange 312a provides similar benefits to that of the circular flange 212a of the trocar tip protector 200, and thus, will not be described in detail herein for purposes of brevity.

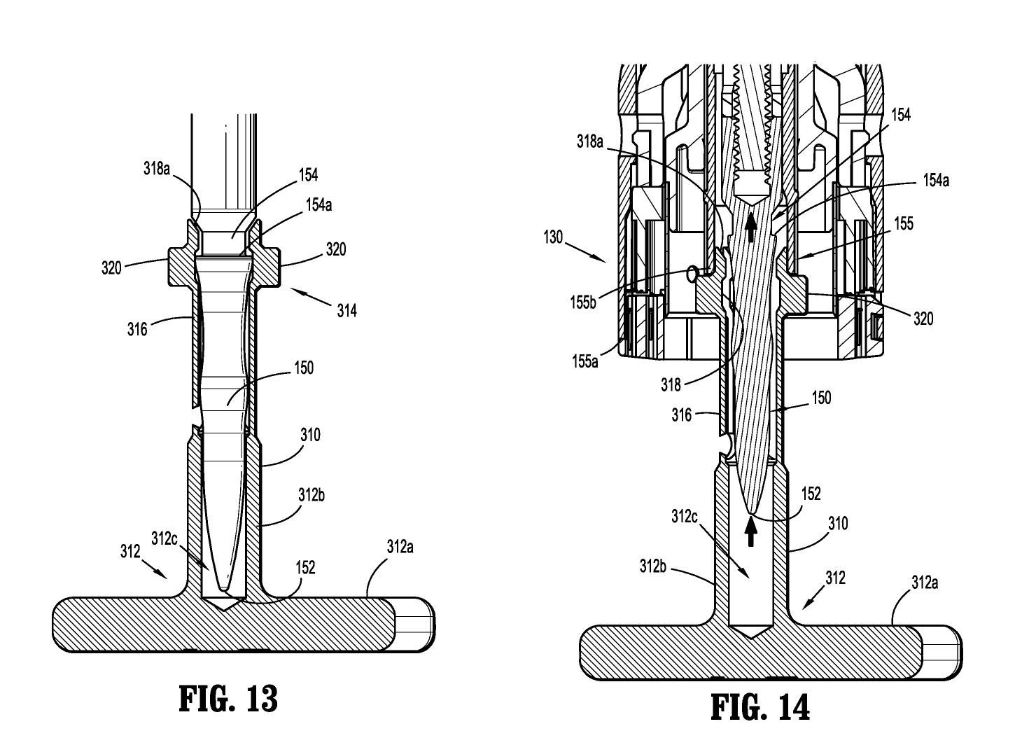

The proximal portion 314 includes a plurality of distally extending legs 316. Each leg of the plurality of legs 316 defines an interior surface 316a (FIG. 12). The interior surface 316a defines an annular groove 318 that is configured and dimensioned to releasably receive an enlarged diameter portion of the trocar 150. When the enlarged diameter portion of the trocar 150 is received within the annular groove 318 of the trocar tip protector 300, the proximal facing lip 154a, which is positioned at one end of the enlarged diameter portion of the trocar 150, abuts the portion of the trocar tip protector 300 defining the proximal end of the annular groove 318. A distal portion of the interior surface 316a of each leg of the plurality of legs 316 includes a bevel or chamfer 316b (FIG. 12). In this manner, as the trocar 150 is advanced within the elongate body 310 of the trocar tip protector 300, each leg of the plurality of legs 316 is urged radially outward (i.e., caused to splay) as the bevel 316b of the legs 316 abuts the trocar 150. When the proximal facing lip 154a of the trocar 150 passes over the annular groove 318 of the trocar tip protector 300 (FIG. 13), the legs 316 of the trocar tip protector 300 will move inwardly to secure the trocar tip protector 300 to the trocar 150. In some embodiments, the proximal and distal ends of the annular groove may define a chamfer 318a to reduce the amount of force required to remove the trocar tip protector 300 from the trocar 150.

In embodiments, the elongate body 310 may include an enlarged through hole 310a interposed between a central portion of adjacent legs of the plurality of legs 316 (FIG. 11). The through hole 310a provides a stress relief as the plurality of legs 316 are caused the splay.

A proximal portion of each leg of the plurality of legs 316 includes a proximal flange 320. In some embodiments, the location of the proximal flange 320 coincides with the location of the annular groove 318 (FIG. 12), although other locations are also contemplated. The proximal flange 320 includes an outer diameter that is greater than the diameter of the proximal end 314 of the plurality of legs 316 and greater than the diameter of an inner portion 155b (FIG. 14) of the housing 155 of the loading unit 130 (FIG. 14). The proximal flange 320 is positioned to engage the inner portion 155b of the housing 155 to inhibit the trocar tip protector 300 from being drawn into the loading unit 130 as the trocar 150 is retracted. Further, as the trocar 150 is retracted into the loading unit 130 with the trocar tip protector 300 attached to the trocar 150, the chamfer 318a of the annular groove 318 abuts the proximal facing lip 154a of the trocar to splay the plurality of legs 316. Simultaneously, the proximal flange 320 on the legs 316 engages the inner portion 155b of the housing of the loading unit 155 to prohibit further retraction of the trocar tip protector 300 and separate the trocar tip protector 300 from the trocar 150. In this manner, the trocar 150 is permitted to retract within the extension assembly 110 without pulling the trocar tip protector 300 into the extension assembly 110 and causing damage to the trocar tip protector 300 and/or the extension assembly 110.

The proximal flange 320 is integrally formed with the plurality of legs 316 (FIG. 12), although it is contemplated that the proximal flange 320 may be adhered or otherwise fastened to the plurality of legs 316 using any suitable means, such as welding, adhesives, fasteners, or the like. In some embodiments, the trocar tip protector 300 may be constructed of any suitable material for use during a sterilization process and may be a resilient material in order to permit deflection of the plurality of legs, such as a metallic, polymeric, or composite material.

Healthcare personnel may attach the trocar tip protector 300 in a similar manner to that of trocar tip protector 200 described above, and therefore, attachment of the trocar tip protector 300 will not be described in detail hereinbelow for purposes of brevity.

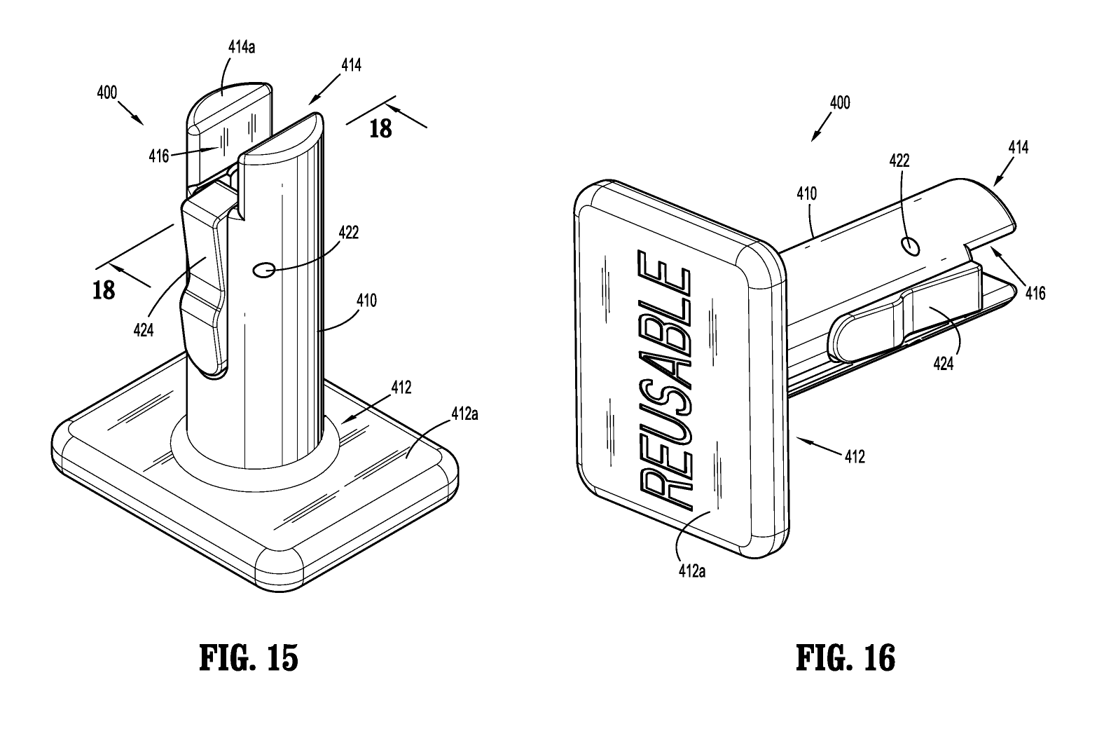

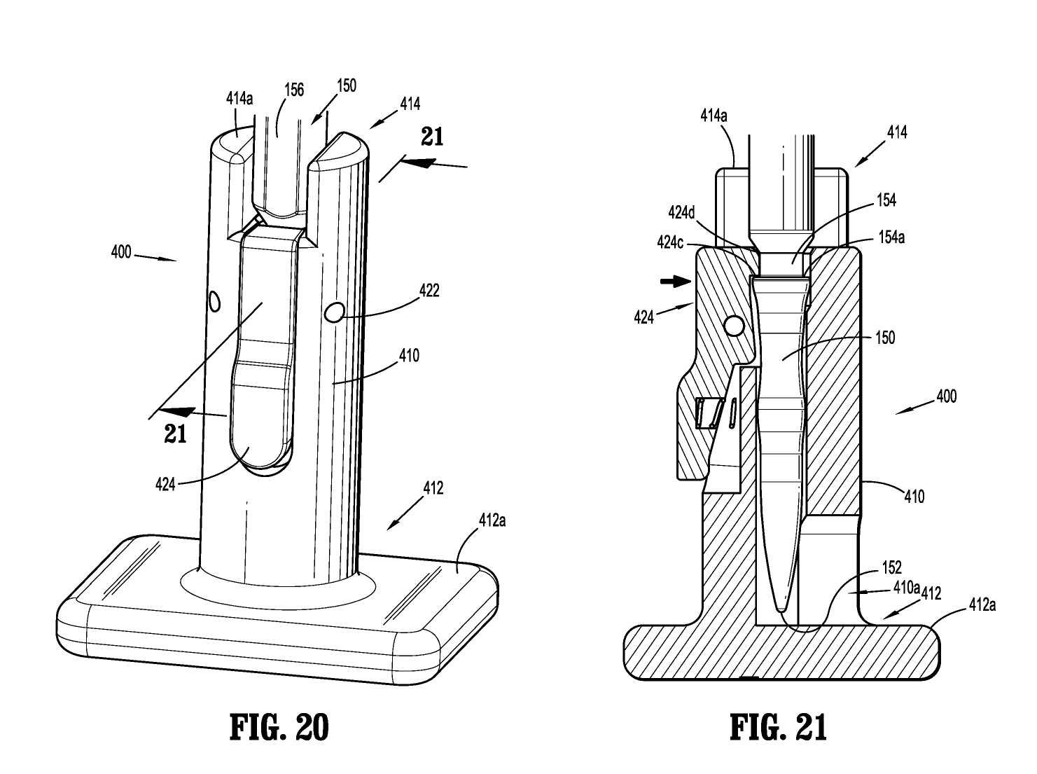

Referring now to FIGS. 15-21, yet another embodiment of a trocar tip protector (protection device) provided in accordance with the present disclosure is illustrated and generally identified by reference numeral 400. Trocar tip protector 400 includes an elongate body 410 including a distal portion 412 and a proximal portion 414. In embodiments, the elongate body 410 may include a circular cross-section although other configurations are envisioned, such as square, rectangular, octagonal, or the like. The distal portion 412 includes a flared proximal end defining a flange 412a having a rectangular shape similar to that of trocar tip protector 300, and thus for purposes of brevity will not be described in detail hereinbelow. However, it is envisioned that flange 412a may include any suitable configuration, such as circular, square, oval, or the like.

A proximal portion of the trocar 150 includes a base portion 156 having a diameter greater than that of the proximal portion of trocar 150 (FIG. 7). The proximal portion 414 of elongate body 410 of trocar tip protector 400 includes a proximal face 414a defining a transverse slot 416 configured to receive the base portion 156 of the trocar 150.

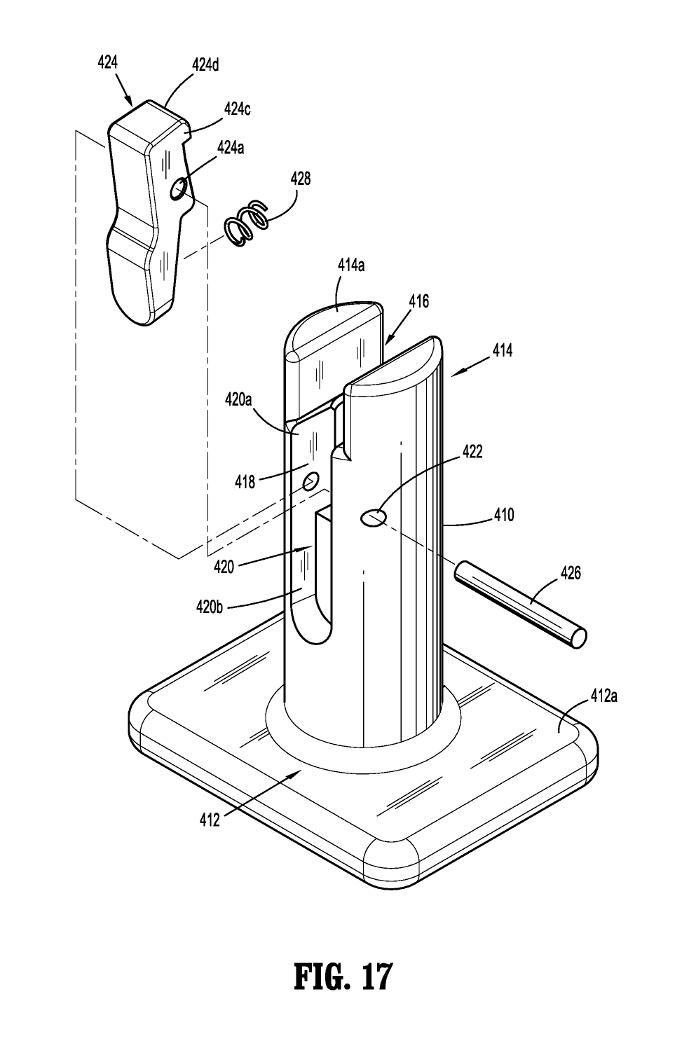

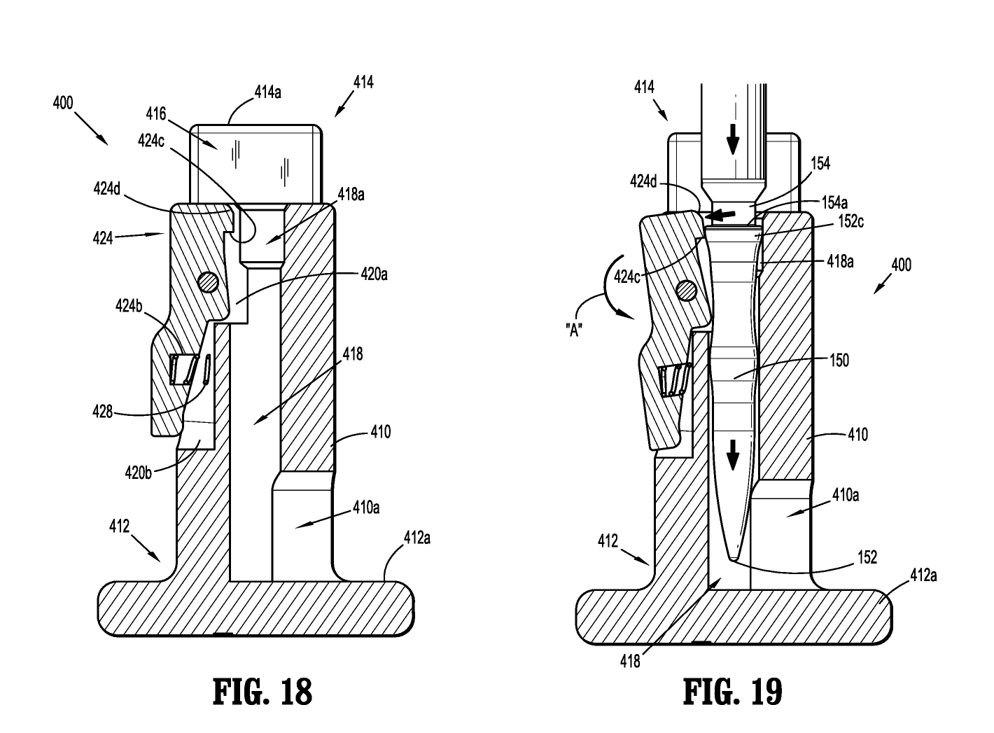

The proximal portion of 414 of the elongate body 410 defines a blind hole 418 (FIG. 18) extending distally from the proximal portion 414 of elongate body 410. The blind hole 418 terminates at the distal portion 412 of the elongate body 410 but does not penetrate the flange 412a (i.e., the blind hole 418 is only open on a proximal end). The blind hole 418 is configured to receive the trocar 150 therein. A proximal portion of the blind hole 418 defines a counterbore 418a that is configured to receive the proximally facing lip 154a of the trocar 150 (FIG. 19). In this manner, the portion of the trocar 150 including the proximal facing lip 154a includes an enlarged diameter portion 152c that has a diameter that is larger than the distal portion of the trocar 150 (FIG. 19). The elongate body 410 includes an outer surface defining a longitudinal slot 420 (FIG. 17). The longitudinal slot 420 includes a generally stepped configuration such that a proximal portion 420a of the longitudinal slot 420 includes a greater depth than a distal portion 420b of the longitudinal slot 420. The outer surface of the elongate body 410 defines a transverse bore 422 extending in a direction perpendicular to that of the transverse slot 416. The transverse bore 422 is generally coincides with the proximal portion 420a of the longitudinal slot 420 (FIG. 17). The transverse bore 422 is configured to receive hinge pin 426.

The trocar tip protector 400 includes a lever 424 configured to be received within the longitudinal slot 420. The lever 424 includes a generally Z-shaped configuration (FIG. 17), although it is envisioned that the lever 424 may include any suitable configuration. A distal portion of the lever 424 includes a through-bore 424a (FIG. 17) dimensioned to receive the hinge pin 426 to rotatably capture the lever 424 within the longitudinal slot 420. A proximal portion of the lever 424 includes a bore 424b (FIG. 18) that is configured to receive a biasing element 428. The biasing element 428 is positioned to act against an inner surface 420b of the longitudinal slot 420 to bias the lever to a first position (i.e., the bore 424b is defined in an inner surface of the lever 424 in a direction normal to the transverse bore 422). An inner surface of the proximal portion of the lever 424 includes a relief 424e (FIG. 18) such that the lever 424 may be rotated about the hinge pin 426 within the longitudinal slot 420.

A proximal portion of the lever 424 defines a barb or ledge 424c (FIG. 18) that is configured to engage the proximal facing lip 154a of the trocar 150 when the lever 424 is in the first position to releasably retain the trocar 150 within the blind hole 418 of the trocar tip protector 400 (FIG. 21). A distal portion of the barb 424c includes a chamfer or fillet 424d that is configured to slidably engage the portion of the trocar 150 including the enlarged diameter portion 152c to allow the trocar 150 to be received within the blind hole 418. During advancement of the trocar 150 into the blind hole 418, the enlarged diameter portion 152c of the trocar 150 abuts the chamfer 424d of the barb 424c and urges the lever 424 to rotate in the direction indicated by “A” (FIG. 19) from a first position, to a second position. A distal portion of the elongate body 410 defines a slot 410a adjacent the flange 412a in open communication with the blind hole 418 to enable the free flow of fluid around the trocar 150 during a sterilization process.

Healthcare personnel may attach the trocar tip protector 400 to a trocar 150 in a similar manner to that of the trocar tip protectors 200 and 300 described above, and therefore, only the differences between the method of attachment of the trocar tip protector 400 and trocar tip protectors 200 and 300 will be detailed herein.

Once the trocar 150 is partially received within the blind hole 418 of the trocar tip protector 400, the trocar 150 is further advanced within the blind hole 418 until the enlarged diameter portion 152c of the trocar 150 abuts the chamfer 424d of the barb 424c of the lever 424 (FIG. 19). Continued advancement of the trocar 150 within the blind hole 418 urges the lever 424 to rotate in the direction indicated by “A” (FIG. 19) from the first position (FIG. 18) to the second position (FIG. 19). The trocar 150 is further advanced within the blind hole 418 until the barb 424c of the lever 424 is received within the annular groove 154 of the trocar 150 and the barb 424c engages the proximally facing lip 154a of the trocar 150 (FIG. 21). The trocar tip protector 400 is dimensioned such that when the barb 424c of the lever 424 is received within the annular groove 154 of the trocar 150 (FIG. 21), the lever 424 is rotated by the biasing member 428 from the second position to the first position (FIG. 21) to releasably secure the trocar tip protector 400 about the trocar 150 such that the pointed distal tip 152 of the trocar 150 is positioned within the elongate body 410 to shield the pointed distal tip 152 of the trocar 150 from accidental contact by healthcare personnel during transport or during a sterilization procedure. In this manner, healthcare personnel are protected from puncture wounds or abrasions that may result from contact with the pointed distal tip 152.

Once the sterilization process is complete, the extension assembly 110, with the trocar tip protector 400 secured thereto, may be removed from the sterilization chamber and packaged or stored until a loading unit 130 is ready to be installed on the distal end of the extension assembly 110 (FIG. 1). At this point, the trocar tip protector 400 may be removed from the trocar 150 to facilitate attachment of the loading unit 130 to the extension assembly 110 (FIG. 1). To remove the trocar tip protector 400 from the trocar 150, healthcare personnel may grasp the flange 412a and depress the proximal portion of the lever 424 of the trocar tip protector 400 to rotate the lever 424 about hinge pin 426 from the first position, to the second position. The proximal portion of the lever 424 is positioned in proximity to the flange 412a such that healthcare personnel may grasp the flange 412a and simultaneously depress the proximal portion of the lever 424 with one hand. The large diameter of the flange 412a of the trocar tip protector 400 provides a greater surface area on which healthcare personnel may grasp when removing the trocar tip protector 400 from the trocar 150. While continuing to depress the proximal portion of the lever 424, the trocar tip protector 400 may be removed from the extension assembly 110 by pulling the trocar tip protector 400 away from the extension assembly 110.

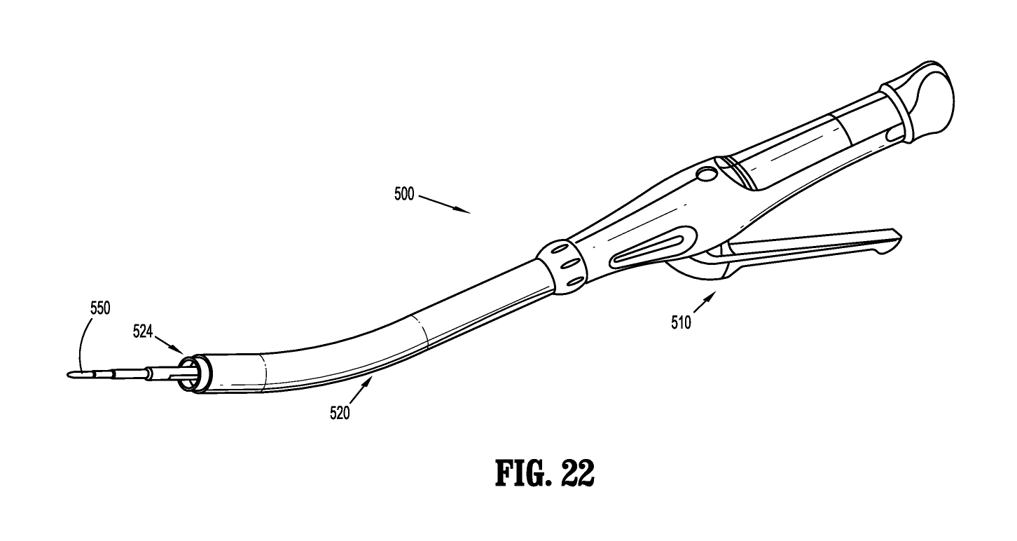

While the trocar tip protectors have been described herein with respect to powered stapling devices, it should of course be understood that the present trocar tip protectors are also suitable for use with manual stapling devices. For example, referring to FIG. 22, a manual surgical stapling device suitable for use with a trocar tip protector in accordance with the present disclosure is illustrated and generally identified by reference numeral 500. Manual surgical stapling device 500 generally includes a handle assembly 510 and an extension assembly 520 extending distally therefrom and terminating in a distal end 524. A trocar 550 is disposed within the extension assembly 520 and extends distally from the extension assembly 520. The trocar 550 is similar to that of the trocar 150 of the extension assembly 110, described above, and therefore, in the interest of brevity, details of the trocar 550 will not be described in detail herein. For a detailed description of the structure and function of an exemplary manual surgical stapling device, reference may be made to U.S. Patent Application Publication No. 2014/0263548, filed Mar. 5, 2014, entitled “surgical Fastener Applying Apparatus,” the content of which is incorporated by reference herein in its entirety. In operation, a trocar tip protector may be selectively secured to the trocar 550 in a similar fashion to the method described above with respect to the powered surgical device 100.

As can be appreciated, trocar tip protectors 200, 300, and 400 may be formed from a suitable material capable of being placed in a sterilization chamber, such as a metallic, polymeric, or composite material and the various components thereof may be formed from the same or different material (i.e., the lever 424 and the elongate body 410 of the trocar tip protector 400 may be formed from the same or different material). Additionally, when each of the trocar tip protectors 200, 300, and 400 are coupled about the trocar 150, a gap exists between the trocar 150 and the respective trocar tip protector 200, 300, or 400 to permit fluid to circulate about the trocar during a sterilization process (FIGS. 10, 13, and 21). In this manner, the trocar tip protectors 200, 300, 400 may remain coupled about the trocar 150 during a sterilization process.

Persons skilled in the art will understand that the devices and methods specifically described herein and illustrated in the accompanying drawings are non-limiting exemplary embodiments. It is envisioned that the elements and features illustrated or described in connection with one exemplary embodiment may be combined with the elements and features of another without departing from the scope of the present disclosure. As well, one skilled in the art will appreciate further features and advantages of the disclosure based on the above-described embodiments. Accordingly, the disclosure is not to be limited by what has been particularly shown and described, except as indicated by the appended claims.

What is claimed is:

1. A protection device, comprising:
an elongate body including a distal portion defining a flange and a proximal portion including a plurality of legs extending proximally from the distal portion, wherein when a distal surface of the flange is positioned on a horizontal surface, the plurality of legs is oriented in a vertical orientation.

2. The protection device of claim 1, wherein the distal portion includes a tubular portion positioned between the flange and the plurality of legs, the tubular portion having an outer surface defining an annular groove, the annular groove configured to allow grasping of the protection device.

3. The protection device of claim 1, wherein a distal surface of the flange includes a depression formed therein.

4. The protection device of claim 1, wherein the protection device is formed form a material suitable for sterilization.

5. The protection device of claim 1, wherein each leg of the plurality of legs terminates in an inwardly extending barb, each barb configured to engage a trocar of a surgical device.

5. The protection device of claim 1, wherein each leg of the plurality of legs has an outer surface including a proximal flange, the proximal flange being dimensioned and configured to inhibit the protection device from being pulled within a surgical device when a trocar of the surgical device is retracted.

6. The protection device of claim 5, wherein each leg of the plurality of legs includes an inner surface defining an annular groove, the annular groove configured to engage a trocar of a surgical device.

7. The protection device of claim 1, wherein the flange is circular.

8. The protection device of claim 1, wherein the flange is rectangular.

9. The protection device of claim 2, wherein the tubular portion defines a bore, the bore extending through at least a portion of the tubular portion.

10. A protection device, comprising:
an elongate body including a distal portion defining a flange and a proximal portion including a proximal face that defines a hole configured to receive a trocar of a surgical device; and
a lever rotatably secured to the elongate body, the lever configured to releasably engage a trocar of a surgical device.

11. The protection device of claim 10, further including a biasing element interposed between the lever and the elongate body, the biasing element biasing the lever into engagement with a trocar of a surgical device.

12. The protection device of claim 10, wherein the flange includes an outer dimension that is larger than the outer dimension of the surgical device.
